(19) United States
(12) Patent Application Publication
(10) Pub. No.: US 2024/0181372 A1
(43) Pub. Date: Jun. 6, 2024

(54) METHODS AND SYSTEMS FOR THE EXTRACTION OF CANNABINOIDS

(71) Applicant: Pobeltsch-Gle AD, Sofia (BG)

(72) Inventor: Ervin Ivanov, Sofia (BG)

(21) Appl. No.: 18/283,676

(22) PCT Filed: Mar. 23, 2022
(86) PCT No.: PCT/IB2022/052630

§ 371 Date: Sep. 22, 2023

Related U.S. Application Data
(60) Provisional application No. 63/165,100, filed on Mar. 23, 2021.

(51) Int. Cl. B01D 11/02; B01D 3/14; B01D 15/08; B01D 61/02; B01D 61/04; C07C 37/00; C07C 51/43; C07C 51/44; C07C 51/47

(52) U.S. Cl. CPC B01D 11/0288; B01D 3/143; B01D 11/0284; B01D 15/08; B01D 61/027; B01D 61/04; C07C 37/002; C07C 51/43; C07C 51/44; C07C 51/47; B01D 2311/04

(57) ABSTRACT

Provided herein are methods and systems that may be used to extract and refine cannabinoids and other natural substances from cannabis plants. The system may mix cannabis plant material with an alkaline water to produce a miscella of solubilized cannabinoids. The system may separate the miscella from the cannabis plant material. The system may acidify the miscella to precipitate the cannabinoids. The system separates the precipitated cannabinoids from the acidic solution. The system extracts on the precipitate to produce raffinate. The raffinate may then be purified and/or an isolated be produced.

### CROSS-REFERENCE TO RELATED APPLICATIONS

This application claims the benefit of U.S. Patent Application No. 63/165,100 filed on Mar. 23, 2021, which is incorporated herein by reference in its entirety.

### TECHNICAL FIELD

The present disclosure relates to methods and systems for the fast, efficient, and cost-effective extraction and refinement of cannabinoids and other natural substances from cannabis plants.

### BACKGROUND

Cannabinoids are a class of compounds found in cannabis plants. Some cannabinoids, including tetrahydrocannabinol (THC) and cannabidiol (CBD), have significant therapeutic and commercial value. Traditional methods of cannabinoid extraction require a drying step to ensure that water does not contaminate the organic solvents used in extraction, or in the case of CO2-mediated extraction, to ensure that CO2 does not react with water to create carbonic acid. However, the drying step itself can be slow, inefficient, expensive, and energy intensive. Additionally, warehousing plants for drying can introduce unwanted contaminants, including mold, bacteria, and vermin. As such, there is high demand for a process of cannabinoid extraction that eliminates the drying step.

The present application overcomes these challenges by providing a rapid, cost-effective, efficient, non-toxic, and environmentally friendly system and method for extracting cannabinoids from cannabis plants. This approach harnesses the use of an alkaline solvent system to solubilize cannabinoids directly from freshly harvested cannabis plants. The disclosed systems and methods are robust enough to be used under a wide range of conditions, including a mobile platform that can process plants in the field.

### SUMMARY

Provided herein are methods and systems of extracting cannabinoids from fresh cannabis plants without the need for a drying step. These methods and systems can be broadly subdivided into four steps: plant extraction, acidification, crude extraction, and refinement.

In some embodiments, the plant extraction step comprises the mixing of cannabis plants, or fractions thereof, with an alkaline solvent. In some embodiments, the alkaline solvent causes cannabinoid acids to ionize, thus making them soluble in water.

In some embodiments, the fractions of a cannabis plant can include, but are not limited to, flowers, buds, leaves, stems, roots, seeds, trichomes, and/or any combination thereof.

In some embodiments, alkaline solvent comprises water. In some embodiments, the water is distilled, deionized, mineral, sparkling, purified, wellwater, seawater, and/or any combination thereof.

In some embodiments, the alkaline solvent has a pH greater than 10. In some embodiments, the pH is 10. In some embodiments, the pH is within the range of 8-10. In some embodiments, the pH is 7. In some embodiments, the pH is within the range of 7-10.

In some embodiments, the alkaline solvent comprises a solvent mixed with one or more alkalis. In some embodiments, the one or more alkalis include, but are not limited to ammonium hydroxide, potassium hydroxide, sodium hydroxide, sodium carbonate, ammonia, sodium acetate, lithium hydroxide, rubidium hydroxide, cesium hydroxide, magnesium hydroxide, calcium hydroxide, strontium hydroxide, barium hydroxide, tetramethylammonium hydroxide, guanidine, alanine, methylamine, and/or any combination thereof.

In some embodiments, the alkaline solvent further comprises one or more buffers. In some embodiments, the one or more buffers include, but are not limited to sodium carbonate, phosphate buffer, mineral buffers, organic buffers, and/or any combination thereof.

In some embodiments, the cannabis plants and alkaline solvent are mixed together for 5-10 minutes. In some embodiments, the cannabis plants and alkaline solvent are mixed together for 10-15 minutes. In some embodiments, the cannabis plants and alkaline solvent are mixed together for 0-5 minutes. In some embodiments, the cannabis plants and alkaline solvent are mixed together for more than 15 minutes. In some embodiments, the length of time the cannabis plants and alkaline solvent are mixed together is dependent on the desired concentration of the cannabinoids.

In some embodiments, the cannabis plants and alkaline solvent are mixed together at ambient temperature. In some embodiments, the cannabis plants and alkaline solvent are mixed together at elevated temperatures. In some embodiments, these elevated temperatures include, but are not limited to, 20-30° ° C., 30-40° ° C., 40-50° C., 50-60° C., 60-70° C., 70-80° ° C., 70-80° ° C., 80-90° C., 90-100° ° C., or greater than 100° C. In some embodiments, the cannabis plants and alkaline solvent are mixed together at a temperature less than 20° C. In some embodiments, the temperature at which the cannabis plants and alkaline solvent are mixed together is dependent on the desired speed of cannabinoid solubilization.

In some embodiments, the cannabis plants and the alkaline solvent are agitated upon mixing. In some embodiments, this agitation includes mechanical agitation, tumbling, stirring, mixing, and/or any combination thereof. In some embodiments, the cannabis plants are mechanically pressed.

In some embodiments, the cannabis plants and the alkaline solvent are sonicated upon mixing.

In some embodiments, after mixing the cannabis plants and alkaline solvent, the product is separated into two fractions; the miscella, comprising the alkaline solvent and solubilized cannabinoids; and the remaining cannabis plant materials.

In some embodiments, the fractions are separated using filtration.

In some embodiments, the remaining cannabis plant materials are subjected to further rounds of extraction. In some embodiments, the remaining cannabis plant materials are pressed to remove any remaining solvent. In some embodiments, the remaining cannabis plant materials are subsequently used as fertilizer or as raw materials for cellulose. In some embodiments, the remaining cannabis plant materials are treated as waste.

In some embodiments, the miscella is reused for one or more further rounds of extraction. In some embodiments, the miscella is reused until it reaches saturation.

In some embodiments, the acidification step comprises mixing the miscella with acid. In some embodiments, the acid reduces the solvent, causing the cannabinoids to precipitate out of solution.

In some embodiments, acid is added to the miscella to lower the pH of the solution to less than 4. In some embodiments, acid is added to the miscella to lower the pH of the solution to within the range of 4-7.

In some embodiments, one or more acids are added to the miscella. In some embodiments, the one or more acids include, but are not limited to Vitamin C, lactic acid, citric acid, hydrochloric acid, sulfuric acid, acetic acid, formic acid, gluconic acid, oxalic acid, tartaric acid, boric acid, chromic acid, hexafluorophosphoric acid, fluoroboric acid, fluoroantimonic acid, phosphoric acid, nitric acid, fluorosulfuric acid, sulfuric acid, hydrofluoric acid, hydrochloric acid, hydrobromic acid, hydroiodic acid, hypochlorous acid, chlorous acid, chloric acid, perchloric acid, hypofluorous acid, and/or any combination thereof.

In some embodiments, the acidification step comprises mixing the miscella with salts. In some embodiments, the salts reduce the solvent, causing the cannabinoids to precipitate out of solution. In some embodiments, both acid and salts are mixed with the miscella.

In some embodiments, salts are added to the miscella to lower the pH of the solution to less than 4. In some embodiments, salts are added to the miscella to lower the pH of the solution to within the range of 4-7.

In some embodiments, one or more salts are added to the miscella. In some embodiments, the one or more salts include, but are not limited to calcium dichloride, calcium sulfate, ferric chloride, and/or any combination thereof.

In some embodiments, one or more buffers are added during the acidification step. In some embodiments, the one or more buffers include, but are not limited to sodium carbonate and/or phosphate buffer.

In some embodiments, the cannabinoids form a precipitate following acidification. In some embodiments, this precipitate is removed from the solvent.

In some embodiments, the precipitate is removed using filters. In some embodiments, the precipitate is removed through centrifugation. In some embodiments, the precipitate is removed through decanting. In some embodiments, the precipitate is removed through sedimentation. In some embodiments, the precipitate is removed through evaporation. In some embodiments, the precipitate is removed through crystallization. In some embodiments, the precipitate is removed through other established methods for removing a precipitate from solution.

In some embodiments, the crude extraction step comprises using a standard extraction process on the precipitate. In some embodiments, the extraction is performed using nonpolar solvents. In some embodiments, the extraction is performed using polar solvents. In some embodiments, the extraction is performed using CO2. In some embodiments, the extraction is performed using pressure and heat.

In some embodiments, the nonpolar solvent used in the extraction step includes, but is not limited to, pentane, hexane, benzene, toluene, propane, heptane, cyclohexane, butane, octane, and/or butadiene.

In some embodiments, the polar solvent used in the extraction step includes, but is not limited to, ethanol, methanol, acetic acid, acetone, ammonia, dimethylsulfoxide, formic acid, n-butanol, and/or n-propanol.

In some embodiments, the crude extraction step results in a refined product defined here as raffinate. In some embodiments, the solvents used for extraction, including nonpolar solvents, polar solvents, and/or CO2 may be recovered and reused in future crude extraction steps.

In some embodiments, the refinement step comprises the methods of processing the raffinate into final products. In some embodiments, these methods include, but are not limited to crystallization and/or chromatography.

In some embodiments, crystallization is used to purify and isolate cannabinoids from the raffinate.

In some embodiments, crystallization is facilitated with one or more organic solvents. In some embodiments, the one or more organic solvents include, but are not limited to pentane, hexane, heptane, octane, and/or methylcyclohexane.

In some embodiments, crystallization results in two products; the crystalized cannabinoid isolate; and the remaining solvent, also known as mother liquor. In some embodiments, these products are separated by evaporation. In some embodiments, these products are separated by filtration. In some embodiments, these products are separated by vacuum filtration. In some embodiments, these products are separated by centrifugation.

In some embodiments, the mother liquor is subjected to chromatography to separate the remaining cannabinoids. In some embodiments, the mother liquor is reused as a solvent in further rounds of crystallization.

In some embodiments, chromatography is used to separate and isolate one or more cannabinoids from the raffinate.

In some embodiments, chromatography is facilitated with one or more organic solvents. In some embodiments, the one or more organic solvents include, but are not limited to water, ethanol, hexane, heptane, cyclohexane, methylcyclohexane, methanol, propanol, pentane, and/or octane.

In some embodiments, the one or more organic solvents are reused in further rounds of chromatography.

In some embodiments, the cannabinoids include, but are not limited to, THC, tetrahydrocannabinolic acid (THCA), CBD, cannabidiolic acid (CBDA), cannabinol, cannabigerol, cannabichromene, cannabicyclol, cannabivarin, tetrahydrocannabinol, tetrahydrocannabivarin, tetrahydrocannabinol, cannabidivarin, cannabichromevarin, cannabigerovarin, cannabigerol monomethyl ether, cannabielsoin, and/or cannabicitran.

In some embodiments, the system comprises a tank for the plant extraction step. In some embodiments, the extraction tank further comprises a conveyer belt or loading device to add cannabis plants. In some embodiments, the extraction tank further comprises an intake valve, nozzle, injector, line, pipe, and/or spigot to add alkaline solvent. In some embodiments, the extraction tank comprises a filter for separating the cannabis plants from the miscella. In some embodiments, the extraction tank further comprises an output valve, line, and/or pipe to remove the miscella. In some embodiments, the extraction tank further comprises a screw transporter for transporting the cannabis plants, solvent and/or miscella out of the extraction tank.

In some embodiments, the extraction tank further comprises a stirring element to mix the cannabis and alkaline solvent. In some embodiments, the extraction tank further comprises a heating element to raise the temperature of the cannabis and alkaline solvent mix.

In some embodiments, the system further comprises a storage/thickening tank for further plant extraction. In some embodiments, the system further comprises an unloading screw to transport cannabis plants, solvent and/or miscella from the extraction tank to the storage/thickening tank. In some embodiments, the storage/thickening tank further comprises an intake valve, nozzle, injector, line, pipe, and/or spigot to add alkaline solvent and/or miscella. In some embodiments, the storage/thickening tank further comprises a filter for separating the plants from the miscella. In some embodiments the storage/thickening tank further comprises an output valve, line, and/or pipe to remove the miscella. In some embodiments, the extraction tank further comprises an unloading screw for transporting the cannabis plants out of the storage/thickening tank.

In some embodiments, the storage/thickening tank further comprises a stirring element to mix the cannabis and alkaline solvent. In some embodiments, the storage/thickening tank further comprises a heating element to raise the temperature of the cannabis and alkaline solvent mix.

In some embodiments, the system further comprises a dewatering press to further remove solvent and/or miscella from the cannabis plants. In some embodiments, the system further comprises an unloading screw to transport cannabis plants from the extraction tank and/or storage/thickening tank to the dewatering press.

In some embodiments, the dewatering press further comprises a filter for separating the cannabis plants from the miscella. In some embodiments the dewatering press further comprises an output valve, line, and/or pipe to remove the miscella.

In some embodiments, the system further comprises a tank to store solvent. In some embodiments, the system further comprises a tank to store miscella. In some embodiments, the same tank is used to store solvent and miscella. In some embodiments, the solvent tank is connected to the extraction tank, the storage/thickening tank, and/or the dewatering press. In some embodiments, the system further comprises one or more pumps to control the flow of solvent and/or miscella between the solvent tank, the extraction tank, the storage/thickening tank, and/or the dewatering press.

In some embodiments, the system is contained on a mobile platform such as a truck. In some embodiments, the mobile platform can be operated in the field during a cannabis harvest.

### BRIEF DESCRIPTION OF THE DRAWINGS

The present disclosure will become better understood from the detailed description and the drawings, wherein:

FIG. 1 illustrates a flow chart describing an example extraction process.

FIG. 2 illustrates an example extraction system.

These drawings in no way limit any changes in form and detail that may be made to the disclosure by one skilled in the art without departing from the spirit and scope of the disclosure.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

The following explanations of terms and methods are provided to better describe the present disclosure and to guide those of ordinary skill in the art in the practice of the present disclosure. As used herein, “comprising” means “including” and it is not intended to mean that the compositions and methods exclude elements that are not recited. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than a trace amount of other ingredients and substantial method steps recited. Embodiments defined by each of these transition terms are within the scope of this invention. The singular forms “a” or “an” or “the” include plural references unless the context clearly dictates otherwise. Thus, for example, a reference to “a compound” includes a plurality of compounds, and a reference to “a molecule” is a reference to one or more molecules. Similarly, reference to “comprising a therapeutic agent” includes one or a plurality of such therapeutic agents. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples. All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All references cited herein are incorporated by reference in their entirety.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties for a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

### Methods for Cannabinoid Extraction

Disclosed herein are methods for cannabinoid extraction, which comprise mixing freshly-harvested cannabis plants with an alkaline solvent to create miscella, and then acidifying this miscella to precipitate the cannabinoids. These novel methods work by using the alkalinity of the solvent to ionize the cannabinoid acids, thus making them soluble in water and allowing them to be extracted from the plants. Because these methods avoid the drying step required in conventional extraction protocols, they possess significant cost and speed advantages, as well as the ability to preserve volatile compounds that would otherwise be evaporated during the drying process.

The disclosed methods comprise four broad steps: plant extraction, acidification, crude extraction, and refinement. In some embodiments, some or all of the refinement step and/or crude extraction step may be omitted, depending on the desired end product.

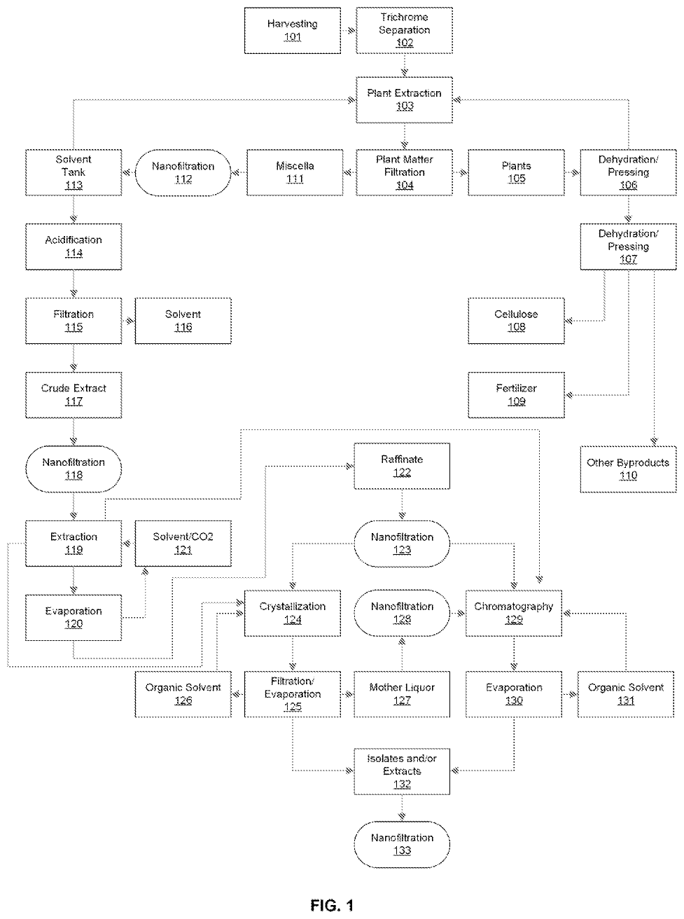

Referring to FIG. 1, the figure describes an example extraction process. Plant material is harvested at a location. The mobile system may be positioned at or near the harvest location, thus allowing the harvested plant material to be processed shortly after harvesting 101. The plant material may be optionally subjected to trichome separation following harvesting 102.

The process may include extraction the plant material 103. Filtering the plant material 104. Creating miscella of the plant material 110 where an alkaline solvent is mixed with the plant material. The miscella in then left for a period of time within the solvent tank 113 for the solvent to affect the plant material. The processed plant material 105 may be later pressed to removed liquid 106. Further the processed plant material may be further dehydrated 107. The remaining processed plant material leaves cellulose 108 which may be deposited back into the environment and be used as fertilizer 109 or as other byproducts 110.

The extraction process in which the cannabis plants are first mixed with an alkaline solvent 103 and subjected to nanofiltration 112 in the solvent tank 113, the filtered miscella is acidified 114, the acidified miscella is then filtrated 115 to produce a crude extract 117 and a recapture of solvent 116. The recaptured solvent may be reintroduced to new batches of plant material for subsequent processing.

The crude extract 117 may be subjected to nanofiltration 118. The filtered crude extract may be extracted to raffinate 119 which may be evaporated 120 with additional processing of a solvent and/or carbon dioxide 121. The raffinate 122 may be further processed to obtain isolates and/or other extracts 133.

The raffinate 122 may be crystallized through a filtration/evaporation process 125 with an organic solvent 126. From this process isolates may be obtained. Furthermore, a mother liquor 127 may be obtained by the filtration/evaporation process 125. The mother liquor may be subjected to nanofiltration 128 and further processed via chromatography 129 where the mother liquor is evaporated and an organic solvent applied, and isolate and/or other extracts are produced. The isolates and other extracts may be subject to nanofiltration to increase concentration 133.

The processes following extraction 119 are optional steps for the further processing of the crude extract inro refined products and may not implemented depending on the desired product. All nanofiltration steps 112, 116, 123, 126, 133 are optional, and can be used to concentrate the products. All processing of leftover plant material 105-110 are optional steps for the production of byproducts and are not required for the extraction of cannabinoids. The trichrome separation step 102 is optional. The use of the solvent tank 113 is optional, as is subjected the miscella to multiple rounds of extraction. The evaporation step 120 is optional.

In some embodiments, the plant extraction step comprises the harvesting of cannabis plants. In some embodiments, the cannabis plants have been previously harvested. In some embodiments, the extraction step is performed within 0-6 hours of harvesting. In some embodiments, the extraction step is performed more than 6 hours after harvesting. In some embodiments, the extraction step is performed before the plants begin to rot.

In some embodiments, the harvested plants are subjected to trichome separation prior to extraction. In some embodiments, the trichomes are separated mechanically. In some embodiments, mechanical separation includes, but is not limited to, agitation, tumbling, grinding, sieving, or any combination thereof. In some embodiments, the trichomes are separated manually. In some embodiments, the trichomes are separated using chilled water.

In some embodiments, the plants are exposed to elevated temperatures following harvesting to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD and/or THCA to THC.

In some embodiments, the plant extraction step comprises the mixing of cannabis plants, or fractions thereof, with an alkaline solvent. In some embodiments, the alkaline solvent causes cannabinoids to ionize, thus making them soluble in water.

In some embodiments, the fractions of a cannabis plant can include, but are not limited to, flowers, buds, leaves, stems, roots, seeds, trichomes, and/or any combination thereof.

In some embodiments, alkaline solvent comprises water. In some embodiments, the water is distilled, deionized, mineral, sparkling, purified, wellwater, seawater, and/or any combination thereof.

In some embodiments, the alkaline solvent has a pH greater than 10. In some embodiments, the pH is 10. In some embodiments, the pH is within the range of 8-10.

In some embodiments, the alkaline solvent comprises a solvent mixed with one or more alkalis. In some embodiments, the one or more alkalis include, but are not limited to ammonium hydroxide, potassium hydroxide, sodium hydroxide, sodium carbonate, ammonia, sodium acetate, lithium hydroxide, rubidium hydroxide, cesium hydroxide, magnesium hydroxide, calcium hydroxide, strontium hydroxide, barium hydroxide, tetramethylammonium hydroxide, guanidine, alanine, methylamine, and/or any combination thereof.

In some embodiments, the cannabis plants are loaded into an empty tank first, and the alkaline solvent is added second. In some embodiments, the cannabis plants are added to a tank pre-filled with alkaline solvent. In some embodiments, the cannabis plants and alkaline solvent are added simultaneously.

In some embodiments, the extraction occurs in sequential tanks, a smaller extraction tank and a larger storage/thickening tank.

In some embodiments, the cannabis plants and alkaline solvent are mixed together for 5-10 minutes. In some embodiments, the cannabis plants and alkaline solvent are mixed together for 10-15 minutes. In some embodiments, the cannabis plants and alkaline solvent are mixed together for 0-5 minutes. In some embodiments, the cannabis plants and alkaline solvent are mixed together for more than 15 minutes. In some embodiments, the length of time the cannabis plants and alkaline solvent are mixed together is dependent on the desired concentration of the cannabinoids. In some embodiments 60-70% of the cannabinoids are extracted upon first contact of the cannabis plants with the alkaline solvent. In some embodiments, more than 90% of the cannabinoids are extracted within 5 minutes of contact of the cannabis plants with the alkaline solvent.

In some embodiments, alkaline solvent is continually removed as fresh solvent is added.

In some embodiments, the cannabis plants and alkaline solvent are mixed together at ambient temperature. In some embodiments, the cannabis plants and alkaline solvent are mixed together at elevated temperatures. In some embodiments, these elevated temperatures include, but are not limited to, 20-30° ° C., 30-40° ° C., 40-50° ° C., 50-60° C., 60-70° C., 70-80° ° C., 70-80° C., 80-90° C., 90-100° C., or greater than 100° C. In some embodiments, the cannabis plants and alkaline solvent are mixed together at a temperature less than 20° C. In some embodiments, the temperature at which the cannabis plants and alkaline solvent are mixed together is dependent on the desired speed of cannabinoid extraction. In some embodiments, higher temperatures increase the speed of extraction.

In some embodiments, high temperatures may convert CBDA to CBD and.or THCA to THC through decarboxylation. In some embodiments, the higher the temperature, the more quickly CBDA will decarboxylate.

In some embodiments, the cannabis plants and the alkaline solvent are agitated upon mixing. In some embodiments, this agitation includes mechanical agitation, tumbling, stirring, mixing, and/or any combination thereof. In some embodiments, the cannabis plants are mechanically pressed. In some embodiments, the mixture is pressurized.

In some embodiments, the cannabis plants and the alkaline solvent are sonicated upon mixing. In some embodiments, sonication increases solubilization.

In some embodiments, after mixing the cannabis plants and alkaline solvent, the product is separated into two fractions; the miscella, comprising the alkaline solvent and solubilized cannabinoids; and the remaining cannabis plant materials.

In some embodiments, the two fractions are separated using filtration. In some embodiments, the two fractions are separated using centrifugation and decantation.

In some embodiments, an unloading screw transports the cannabis plants out of the tank and into a dewatering press.

In some embodiments, the remaining cannabis plant materials are subjected to further rounds of extraction. In some embodiments, the remaining cannabis plant materials are pressed and/or dried to remove any remaining solvent. In some embodiments, the remaining cannabis plant materials are subsequently used as fertilizer or as raw materials for cellulose. In some embodiments, the remaining cannabis plant materials are treated as waste.

In some embodiments, the miscella is reused for one or more further rounds of extraction. In some embodiments, the miscella is reused until it reaches saturation. In some embodiments, more freshly harvested cannabis plants may be mixed with the miscella to reach saturation. In some embodiments, fresh alkaline solvent is used for further rounds of extraction.

In some embodiments, the saturation point of the miscella is dependent on the concentration of both cannabinoids and ballast substances in the plants. In some embodiments, some plants with more ballast substances may have a lower saturation point.

In some embodiments, the miscella is subjected to nanofiltration to increase its concentration. In some embodiments, the nanofiltration is performed using a membrane with pore sizes of 1-10 nanometers. In some embodiments, the pore sizes are smaller than 1 nanometer. In some embodiments, the pore sizes are larger that 10 nanometers. In some embodiments, the pore density is 1-100 pore per cm². In some embodiments, the pore density is less than 1 per cm². In some embodiments, the pore density is greater than 100 per cm². In some embodiments, the solute is transported across the membrane through diffusion, convection, electromigration, or any combination thereof.

In some embodiments, the miscella is exposed to elevated temperatures following extraction to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD and/or THCA to THC.

In some embodiments, the acidification step comprises mixing the miscella with one or more acids. In some embodiments, the one or more acids reduce the solvent, causing the cannabinoids to precipitate out of solution.

In some embodiments, the miscella is loaded into an empty tank first, and the one or more acids are added second. In some embodiments, the miscella is added to a tank pre-loaded with one or more acids. In some embodiments, the miscella and the one or more acids are added simultaneously.

In some embodiments, the one or more acids are mixed with the miscella to lower the pH of the solution to less than 4. In some embodiments, the one or more acids are mixed with the miscella to lower the pH of the solution to within the range of 4-7.

In some embodiments, the one or more acids include, but are not limited to Vitamin C, lactic acid, citric acid, hydrochloric acid, sulfuric acid, acetic acid, formic acid, gluconic acid, oxalic acid, tartaric acid, boric acid, chromic acid, hexafluorophosphoric acid, fluoroboric acid, fluoroantimonic acid, phosphoric acid, nitric acid, fluorosulfuric acid, sulfuric acid, hydrofluoric acid, hydrochloric acid, hydrobromic acid, hydroiodic acid, hypochlorous acid, chlorous acid, chloric acid, perchloric acid, and/or hypofluorous acid.

In some embodiments, the miscella and the one or more acids are mixed together for 0-5 minutes. In some embodiments, the miscella and the one or more acids are mixed together for 5-10 minutes. In some embodiments, the miscella and the one or more acids are mixed together for 10-15 minutes. In some embodiments, the miscella and the one or more acids are mixed together for more than 15 minutes.

In some embodiments, the miscella and the one or more acids are mixed together at ambient temperature. In some embodiments, the miscella and the one or more acids are mixed together at elevated temperatures. In some embodiments, these elevated temperatures include, but are not limited to, 20-30° C., 30-40° ° C., 40-50° C., 50-60° ° C., 60-70° C., 70-80° C., 70-80° ° C., 80-90° C., 90-100° C., or greater than 100° C. In some embodiments, the miscella and the one or more acids are mixed together at a temperature less than 20° C.

In some embodiments, the cannabinoids form a precipitate following acidification. In some embodiments, this precipitate is removed from the acidified solvent.

In some embodiments, the precipitate is removed using filters. In some embodiments, the precipitate is removed using pressurized filtration. In some embodiments, the precipitate is removed through centrifugation. In some embodiments, the precipitate is removed through decanting. In some embodiments, the precipitate is removed through sedimentation. In some embodiments, the precipitate is removed through evaporation. In some embodiments, the precipitate is removed through crystallization. In some embodiments, the precipitate is removed through other established methods for removing a precipitate from solution.

In some embodiments, a 1-micron filter is used to remove the precipitate. In some embodiments, a filter smaller than 1-micron is used to remove the precipitate. In some embodiments, a filter larger than 1-micron is used to remove the precipitate.

In some embodiments, the filtered precipitate is subjected to nanofiltration to increase its concentration. In some embodiments, the nanofiltration is performed using a membrane with pore sizes of 1-10 nanometers. In some embodiments, the pore sizes are smaller than 1 nanometer. In some embodiments, the pore sizes are larger that 10 nanometers. In some embodiments, the pore density is 1-100 pore per cm². In some embodiments, the pore density is less than 1 per cm². In some embodiments, the pore density is greater than 100 per cm². In some embodiments, the solute is transported across the membrane through diffusion, convection, electromigration, or any combination thereof.

In some embodiments, the precipitate is exposed to elevated temperatures following acidification to convert CBDA to CBD and/or THCA to THC through decarboxylation. In some embodiments, the temperature is elevated to 88-200° C. In some embodiments, the temperature is elevated above 200° C. In some embodiments, higher temperatures are associated with a faster conversion of CBDA to CBD and/or THCA to THC.

In some embodiments, the crude extraction step comprises using a standard extraction process on the precipitate. In some embodiments, the extraction is performed using nonpolar solvents. In some embodiments, the extraction is performed using polar solvents. In some embodiments, the extraction is performed using CO2. In some embodiments, the extraction is performed using pressure and heat.

In some embodiments, the nonpolar solvent used in the extraction step includes, but is not limited to, pentane, hexane, benzene, toluene, propane, heptane, cyclohexane, butane, octane, and/or butadiene.

In some embodiments, the polar solvent used in the extraction step includes, but is not limited to, ethanol, methanol, acetic acid, acetone, ammonia, dimethylsulfoxide, formic acid, n-butanol, and/or n-propanol.

In some embodiments, the crude extraction step results in a refined extract, defined here as raffinate. In some embodiments, the solvents used for extraction, including nonpolar solvents, polar solvents, and/or CO2 may be recovered and reused in future crude extraction steps.

In some embodiments, the raffinate is subjected to nanofiltration to increase its concentration. In some embodiments, the nanofiltration is performed using a membrane with pore sizes of 1-10 nanometers. In some embodiments, the pore sizes are smaller than 1 nanometer. In some embodiments, the pore sizes are larger that 10 nanometers. In some embodiments, the pore density is 1-100 pore per cm². In some embodiments, the pore density is less than 1 per cm². In some embodiments, the pore density is greater than 100 per cm². In some embodiments, the solute is transported across the membrane through diffusion, convection, electromigration, or any combination thereof.

In some embodiments, the raffinate is exposed to elevated temperatures following extraction to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD and/or THCA to THC.

In some embodiments, the refinement step comprises the methods of processing the raffinate into final products. In some embodiments, these methods include, but are not limited to crystallization and/or chromatography.

In some embodiments, crystallization is used to purify and isolate cannabinoids within the raffinate.

In some embodiments, crystallization is facilitated with one or more organic solvents. In some embodiments, the one or more organic solvents include, but are not limited to pentane, hexane, heptane, octane, and/or methylcyclohexane.

In some embodiments, crystallization results in two products; the crystalized cannabinoid isolate; and the remaining solvent, also known as mother liquor. In some embodiments, these products are separated by evaporation. In some embodiments, these products are separated by filtration. In some embodiments, these products are separated by vacuum filtration. In some embodiments, these products are separated by centrifugation.

In some embodiments, the products are exposed to elevated temperatures following crystallization to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD and/or THCA to THC.

In some embodiments, the mother liquor is subjected to nanofiltration to increase its concentration. In some embodiments, the nanofiltration is performed using a membrane with pore sizes of 1-10 nanometers. In some embodiments, the pore sizes are smaller than 1 nanometer. In some embodiments, the pore sizes are larger that 10 nanometers. In some embodiments, the pore density is 1-100 pore per cm². In some embodiments, the pore density is less than 1 per cm². In some embodiments, the pore density is greater than 100 per cm². In some embodiments, the solute is transported across the membrane through diffusion, convection, electromigration, or any combination thereof.

In some embodiments, the mother liquor is exposed to elevated temperatures to convert CBDA to CBD and/or THCA to THC through decarboxylation. In some embodiments, the temperature is elevated to 88-200° C. In some embodiments, the temperature is elevated above 200° ° C. In some embodiments, higher temperatures are associated with a faster conversion of CBDA to CBD and/or THCA to THC.

In some embodiments, the mother liquor is subjected to chromatography to separate the remaining cannabinoids. In some embodiments, the mother liquor is reused as a solvent in further rounds of crystallization.

In some embodiments, chromatography is used to separate and isolate one or more cannabinoids and/or other extracts from the raffinate.

In some embodiments, chromatography is facilitated with one or more organic solvents. In some embodiments, the one or more organic solvents include, but are not limited to water, ethanol, hexane, heptane, cyclohexane, methylcyclohexane, methanol, propanol, pentane, and/or octane.

In some embodiments, the one or more organic solvents are reused in further rounds of chromatography.

In some embodiments, the isolated and/or other extracts are subjected to nanofiltration to increase their concentration. In some embodiments, the nanofiltration is performed using a membrane with pore sizes of 1-10 nanometers. In some embodiments, the pore sizes are smaller than 1 nanometer. In some embodiments, the pore sizes are larger that 10 nanometers. In some embodiments, the pore density is 1-100 pore per cm². In some embodiments, the pore density is less than 1 per cm². In some embodiments, the pore density is greater than 100 per cm². In some embodiments, the solute is transported across the membrane through diffusion, convection, electromigration, or any combination thereof.

In some embodiments, the isolates and/or extracts are exposed to elevated temperatures to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD and/or THCA to THC.

In some embodiments, the cannabinoids include, but are not limited to, THC, THCA, CBD, CBDA, cannabinol, cannabigerol, cannabichromene, cannabicyclol, cannabivarin, tetrahydrocannabinol, tetrahydrocannabivarin, tetrahydrocannabinol, cannabidivarin, cannabichromevarin, cannabigerovarin, cannabigerol monomethyl ether, cannabielsoin, and/or cannabicitran.

In some embodiments, the temperature is elevated at any step within the method to convert CBDA to CBD and/or THCA to THC through decarboxylation. In some embodiments, the temperature is elevated to 88-200° ° C. In some embodiments, the temperature is elevated above 200° C. In some embodiments, higher temperatures are associated with a faster conversion of CBDA to CBD.

### Systems for Cannabinoid Extraction

Also provided herein are systems for cannabinoid extraction. The systems work by providing a framework for solubilizing the cannabinoids in an alkaline solvent, producing miscella. Because this approach requires no lengthy, energy-intensive drying step, it is possible to enable these systems within a mobile platform, such that cannabis plants could be processed in the field concurrent with the harvest.

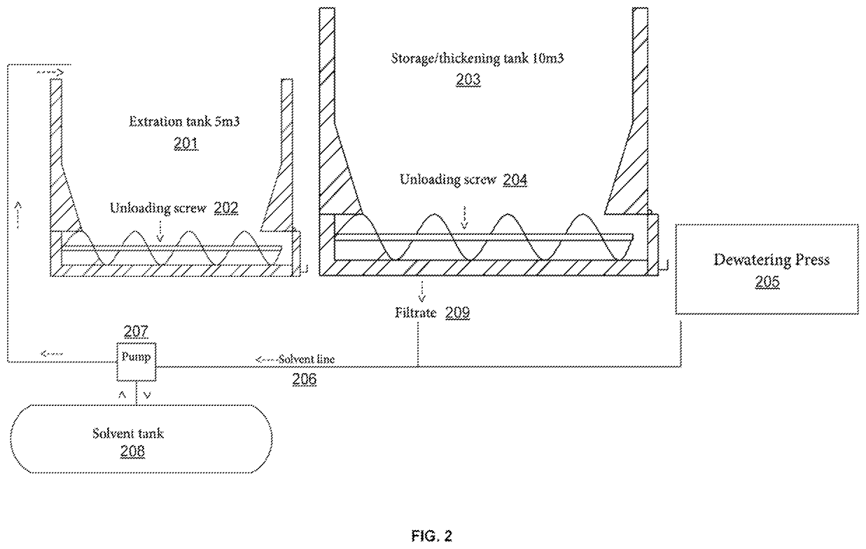

Referring to FIG. 2, the figure illustrates a schematic representation of an extraction system in which a first tank 201 is used for extraction and separation of the miscella from the cannabis plant material, and a second tank 203 is used for further storage and separation of the miscella from the cannabis plant material. A dewatering press 205 is used to extract any remaining water or miscella from the remaining plant material. An unloading screw 202, 204 is used to transfer the plant matter from the extraction tank to the storage tank and then to the dewatering press. A solvent tank 208 with a pump 207 used to collect, store, and transport solvent and miscella 209 to and from the tanks. Solvent and/or unsaturated miscella is added to the extraction tank, and is removed from the storage/thickening tank and the dewatering press.

The disclosed systems comprise two primary elements: a system for solubilizing the cannabinoids, and a system for the separation of the miscella from the remaining plant matter. In some embodiments, the systems further comprise a system for the acidification of the miscella. In some embodiments, the systems further comprise a system for the extraction of raffinate from the acidified miscella precipitate. In some embodiments, the systems further comprise a system for the crystallization of the raffinate. In some embodiments, the systems further comprise a system for the chromatography of the raffinate.

In some embodiments, the system comprises an extraction tank for the plant extraction. In some embodiments, the size of the extraction tank is dependent on the volume of cannabis to be extracted. In some embodiments, the extraction tank is 5m3. In some embodiments, the extraction tank is less than 5m3. In some embodiments, the extraction tank is greater than 5m3.

In some embodiments, the extraction tank further comprises a conveyer belt or loading device to add cannabis plants to the system.

In some embodiments, the extraction tank further comprises an intake valve, nozzle, injector, line, pipe, and/or spigot to add alkaline solvent. In some embodiments, the extraction tank further comprises an output valve, line, and/or pipe to remove the miscella. In some embodiments, the input and output line are the same.

In some embodiments, the extraction tank further comprises a filter for separating the cannabis plants from the miscella. In some embodiments, the extraction tank further comprises a net for separating the cannabis plants from the miscella.

In some embodiments, the extraction tank further comprises a stirring element.

In some embodiments, the extraction tank further comprises a heating element. The heating element may be used to increase the temperature of the solvent and/or miscella.

In some embodiments, the extraction tank further comprises an ultrasonicator. The ultrasonicator introduces sonic vibrations to the alkaline solvent to causing agitation to the solvent and/or miscella.

In some embodiments, the extraction tank further comprises a sealable hatch to pressurize the tank. The tank may be further pressurized by introducing air into the tank, where the pressure is greater than the ambient pressure outside of the tank.

In some embodiments, the system further comprises a storage/thickening tank. In some embodiments, the size of the extraction tank is dependent on the volume of cannabis to be extracted. In some embodiments, the extraction tank is 10 m3. In some embodiments, the extraction tank is less than 10 m3. In some embodiments, the extraction tank is greater than 10 m3. In some embodiments, the storage/thickening tank is larger than the extraction tank.

In some embodiments, the system further comprises an unloading screw to transport the cannabis plants, solvent and/or miscella from the extraction tank to a storage/thickening tank.

In some embodiments, the storage/thickening tank further comprises an intake valve, nozzle, injector, line, pipe, and/or spigot to add alkaline solvent. In some embodiments, the storage/thickening tank further comprises an output valve, line, and/or pipe to remove the miscella. In some embodiments, the input and output line are the same.

In some embodiments, the storage/thickening tank further comprises a filter for separating the cannabis plants from the miscella. In some embodiments, the storage/thickening tank further comprises a net for separating the cannabis plants from the miscella.

In some embodiments, the storage/thickening tank further comprises a stirring element. The stirring element may be used to stir the solvent and/or miscella.

In some embodiments, the storage/thickening tank further comprises a heating element.

In some embodiments, the storage/thickening tank further comprises an ultrasonicator.

In some embodiments, the storage/thickening tank further comprises a sealable hatch to pressurize the tank.

In some embodiments, the system further comprises a dewatering press to further remove solvent from the remaining cannabis. In some embodiments, the system further comprises a heating element to dry the remaining cannabis.

In some embodiments, the system further comprises an unloading screw to transport the cannabis plants from the storage/thickening tank to the dewatering press.

In some embodiments, the dewatering press further comprises a filter for separating the plants from the miscella. In some embodiments the dewatering press further comprises an output valve, line, and/or pipe to remove the miscella.

In some embodiments, the system further comprises one or more solvent tanks to store the alkaline solvent. In some embodiments, the system further comprises one or more miscella tanks. In some embodiments, the solvent tank and the miscella tank are the same.

In some embodiments, the one or more solvent tank and/or miscella tanks are connected to the extraction tank, the storage/thickening tank, and/or the dewatering press. In some embodiments, one or more pumps are used to control the flow of solvent and/or miscella between the solvent tank, the extraction tank, the storage/thickening tank, and/or the dewatering press.

In some embodiments, the connections between tanks comprise pipes and/or hoses. In some embodiments, these connections further comprise operable valves to control the flow to each tank.

In some embodiments, the alkaline solvent comprises water. In some embodiments, the water is distilled, deionized, mineral, sparkling, purified, wellwater, seawater, and/or any combination thereof.

In some embodiments, the alkaline solvent has a pH greater than 10. In some embodiments, the pH is 10. In some embodiments, the pH is within the range of 8-10.

In some embodiments, the alkaline solvent comprises a solvent mixed with one or more alkalis. In some embodiments, the one or more alkalis include, but are not limited to ammonium hydroxide, potassium hydroxide, sodium hydroxide, sodium carbonate, ammonia, sodium acetate, lithium hydroxide, rubidium hydroxide, cesium hydroxide, magnesium hydroxide, calcium hydroxide, strontium hydroxide, barium hydroxide, tetramethylammonium hydroxide, guanidine, alanine, methylamine, and/or any combination thereof.

In some embodiments, the system is contained on a mobile platform such as a truck. In some embodiments, the mobile platform can be operated in the field during a cannabis harvest. In some embodiments, the system is immobile.

The disclosed systems and methods overcomes limitations inherent in other extraction processes. The drying step, a requirement in traditional approaches, can be slow, energy-intensive, and expensive. Moreover, warehousing a large-scale crop for drying, can introduce unwanted contaminants, including mold, bacteria, and vermin.

The disclosed systems and methods, on the other hand does not require a drying step, thus offering several advantages over traditional methods. Alkaline solvents, used to solubilize the cannabinoids, are inexpensive and work almost instantaneously, a vast improvement over the drying process. Furthermore, the use of alkaline solvents allows the recovery of volatile compounds, such as terpenes, which are normally evaporated during the drying process. Terpenes, which have a distinctive aroma, have long been a signifier of quality in cannabinoid products, and can increase the value of the extracts. Additionally, because the disclosed systems and methods do not require the elevated temperatures of the drying process, it allows for the purification of CBDA. While CBDA will eventually decarboxylate into CBD, this process takes time, and can potentially double the shelf life of the extract.

Another added benefit is the portability of the method. Given the speed with which alkaline solvents solubilize the cannabinoids, freshly harvested cannabis plants can be processed in the field through the use of a mobile platform. This avoids the added costs of warehousing imposed by the drying step. Moreover, the leftover plant material following the extraction process can be used as fertilizer.

The disclosed systems and methods are also robust, and can be operated at any practical scale or temperature. Additionally, it has the added benefit of being environmentally friendly and non-toxic, as all of the compounds involved are safe for human consumption.

### Practical Applications

The disclosed systems and methods can be used to extract multiple cannabinoids, including such high-value targets as THC and CBD. Furthermore, the disclosed systems and methods provides a system that can be used as a mobile platform in the field to process fresh cannabis plants during the harvest. The mobile platform may be moved to a physical location to where the plants are harvested. This allows faster processing of harvested matter which typically has to be transported to an off-site location where the material is chemically processed. The longer the platter matter sits without being processed, reduces the overall yield of THC and CBD.

### EXAMPLES

### Example 1: Mobile Extraction of Miscella from Freshly Harvested Cannabis Plants

A half-acre of cannabis plants are harvested and loaded in batches into a 5 m3 extraction tank on a mobile extraction truck. An extraction tank is simultaneously filled with pH 10 water, pumped in from the solvent tank. The alkaline water extracts the cannabinoids from the plants, with almost 60-70% of the total cannabinoids extracted at the moment of first contact. The alkaline water, as it solubilizes the cannabinoids, becomes a miscella. An unloading screw slowly transports the cannabis plants, alkaline water, and miscella from the tank and into a 10 m3 storage/thickening tank.

The storage/thickening tank is slowly filled with the cannabis plants, alkaline water, and miscella. As it fills, the pressure within the tank rises, forcing the alkaline water and miscella through a 1-micron filter and into an exit line. The exit line is connected to the solvent tank, which in turn is pumped back into the extraction tank. The concentration of cannabinoids within this closed loop slowly increases.

After 10 minutes of exposure to the alkaline solvent, the unloading screw transports cannabis plants, along with a limited amount of solvent and/or miscella, into a dewatering press. The cannabis plants are pressed, and any remaining solvent and/or miscella is passed through a 1-micron filter and into an exit line. The exit line is also connected to the solvent tank. After pressing, the cannabis plants are piled on the ground, and will later be distributed on the fields as fertilizer.

After all of the cannabis plants are added, the miscella within the system is collected into drums to be used in downstream processing.

### Example 2: CBD Extraction from a Flower of Single Cannabis Plant

The flowers of a cannabis plant are picked and added into a 1 L contained of pH 10 water. The solution is stirred for 5 minutes, and the flowers are separated from the miscella via filtration.

The miscella is mixed with citric acid to a final pH of 4. The mixture is slowly stirred for 5 minutes. The acidity causes the cannabinoids to precipitate out of solution. Using a 1-micron filter, the precipitate is separated from the rest of the solution.

A standard CO2 extraction is performed on the precipitate. The precipitate is exposed to pressurized supercritical CO2, which carries cannabis oil particles to a cyclonic separator. The lower pressure of the separator causes the CO2 and the cannabis oil to separate. The cannabis oil is collected as a purified extract defined here as raffinate.

The raffinate is passed through a chromatographic column and separated into fractions. The pure, THC-free CBD is collected and bottled for storage.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

1-34. (canceled)

35. A method of cannabinoid acids and/or cannabinoids extraction, the method comprising:
mixing an amount of alkaline solvent with a first amount of cannabis plant material thereby producing a miscella in a tank, wherein the alkaline solvent has a pH>10;
reusing the miscella for mixing with one or more additional amounts of cannabis plant material;
mixing the miscella with an acid to produce an acidic solution, wherein the acidic solution causes cannabinoid acids and/or cannabinoids to precipitate from the miscella; and
filtering the precipitate to produce an amount of a crude extract and an amount of the alkaline solvent.

36. The method of claim 35, wherein the alkaline solvent is mixed with the first amount of cannabis plant material for between 10 and 15 minutes.

37. The method of claim 35, wherein the acid is mixed with the miscella for below 10 minutes.

38. The method of claim 35, wherein the alkaline solvent and the first amount of cannabis plant material are agitated while mixing.

39. The method of claim 35, further comprising the step:
separating the cannabis plant material to remove the alkaline solvent from the cannabis plant material.

40. The method of claim 35, wherein the miscella is reused until the miscella reaches saturation of the cannabinoid acids and/or cannabinoids.

41. The method of claim 35, further comprising:
performing nanofiltration of the miscella using a membrane with pore sizes less than 10 nanometers.

42. The method of claim 35, further comprising:
performing crystallisation to separate and isolate one or more of the cannabinoid acids and/or cannabinoids from the crude extract.

43. The method of claim 35, further comprising:
performing chromatography to separate and isolate one or more cannabinoid acids and/or cannabinoids from the crude extract.

44. The method of claim 35, further comprising:
performing distillation to separate and isolate one or more of the cannabinoid acids and/or cannabinoids from the crude extract.

45. The method of claim 35, further comprising:
extracting the crude extract to a raffinate.

46. The method of claim 45, further comprising:
performing nanofiltration to purify one or more of the cannabinoids from the raffinate.

47. The method of claim 46, further comprising:
performing chromatography to separate and isolate one or more cannabinoid acids and/or cannabinoids from the raffinate.

48. The method of claim 46, further comprising:
performing crystallisation to separate and isolate one or more of the cannabinoid acids and/or cannabinoids from the raffinate.

49. The method of claim 35, further comprising:
exposing the miscella and crude extract to temperatures ranging from 88-200° C. to convert cannabinoid acids into cannabinoids including cannabidiolic acid (CBDA) to (cannabidiol) CBD and/or tetrahydrocannabinolic acid (THCA) to tetrahydrocannabinol (THC).

50. The method of claim 49, further comprising:
performing crystallisation to purify and isolate one or more cannabinoid acids and/or cannabinoids from the decarboxylated crude extract.

51. A method of cannabinoid acids and/or cannabinoids extraction, the method comprising:
mixing an amount of alkaline solvent with a first amount of cannabis plant material thereby producing a miscella in a tank, wherein the alkaline solvent is alkaline water having a pH>10;
performing nanofiltration of the miscella using a membrane with pore sizes less than 10 nanometers;
reusing the miscella for mixing with one or more additional amounts of cannabis plant material, wherein the miscella is reused until the miscella reaches saturation of the cannabinoid acids and/or cannabinoids;
mixing the nanofiltered miscella with an acid to produce an acidic solution, wherein the acidic solution causes cannabinoid acids and/or cannabinoids to precipitate from the miscella;
separating the cannabis plant material to remove the alkaline solvent from the cannabis plant material; and
filtering the precipitate to produce an amount of a crude extract and an amount of the alkaline solvent.

52. The method of claim 51, further comprising the steps of:
exposing the miscella to temperatures ranging from 88-200° C. to convert cannabinoid acids to cannabinoids including cannabidiolic acid (CBDA) to (cannabidiol) CBD and/or tetrahydrocannabinolic acid (THCA) to tetrahydrocannabinol (THC).

53. The method of claim 51, further comprising:
performing chromatography to separate and isolate one or more cannabinoid acids and/or cannabinoids from the crude extract.

54. The method of claim 51, further comprising:
performing crystallisation to separate and isolate one or more of the cannabinoid acids and/or cannabinoids from the crude extract.
